(19) United States
(12) Patent Application Publication
(10) Pub. No.: US 2025/0188404 A1
(43) Pub. Date: Jun. 12, 2025

(54) CULTURE CAPSULE AND METHOD FOR RESUSCITATING BACTERIA IN VIABLE BUT NON-CULTURABLE (VBNC) STATE

(71) Applicant: Innovation Center of Yangtze River Delta, ZJU, Jiaxing City (CN)

(72) Inventors: Xinyu Liao, Zhejiang (CN); Yu Zhao, Zhejiang (CN); Tian Ding, Zhejiang (CN)

(21) Appl. No.: 18/586,997

(22) Filed: Feb. 26, 2024

(30) Foreign Application Priority Data
Dec. 6, 2023 (CN) 202311666404X

(51) Int. Cl. C12N 1/04; C12N 1/20

(52) U.S. Cl. CPC C12N 1/04; C12N 1/20

(57) ABSTRACT

Disclosed is a culture capsule and method for resuscitating bacteria in a viable but non-culturable (VBNC) state, which belongs to the technical field of microbial culture and detection. The present disclosure provides use of a nutrient in the preparation of a resuscitating agent for resuscitating bacteria in a viable but non-culturable (VBNC) state, where the nutrient includes egg yolk lecithin and/or cholesterol.

### CROSS REFERENCE TO RELATED APPLICATION

This patent application claims the benefit and priority of Chinese Patent Application No. 202311666404X filed with the China National Intellectual Property Administration on Dec. 6, 2023, the disclosure of which is incorporated by reference herein in its entirety as part of the present application.

### TECHNICAL FIELD

The present disclosure belongs to the technical field of microbial culture and detection, and specifically relates to a culture capsule and method for resuscitating bacteria in a viable but non-culturable (VBNC) state.

### BACKGROUND

A special dormant state of bacteria usually refers to a dormant state in which bacteria are non-culturable under environmental conditions adverse to survival of bacteria, namely, a VBNC state. A large amount of substantial evidence has shown that most bacteria can enter a special dormant state or show a reduced activity under environmental conditions unfavorable for growth and proliferation of bacteria to escape impacts of the unfavorable environmental conditions. The resuscitation of bacteria in a special dormant state is a complicated process, which cannot be implemented by simply removing environmental stress factors, but sometimes requires the addition of a nutrient, a chemical, or a food matrix.

Chinese patent application CN 115537365A (application date: 2022 Dec. 1, and publication date: 2022 Dec. 30) discloses a resuscitation medium and a method for detecting VBNC-state Acetobacter aceti in food. The resuscitation medium is prepared as follows: an Asaia (AS) medium is first prepared, and then Eagle's Minimum Essential Medium (EMEM) and/or coconut water are/is added to the AS medium, where a volume fraction of the EMEM is 25% to 50%, a volume fraction of the coconut water is 25% to 50%, or a total volume fraction of the EMEM and the coconut water is 25% to 50%. A food sample can be added to the resuscitation medium and cultivated under shaking, such that VBNC-state Asaia spp. in the food sample can be resuscitated and a growth ability of the VBNC-state Asaia spp. can be restored. It can be seen that many of the current media capable of “awakening” bacteria in a special dormant state have a complicated composition and need to be prepared by a cumbersome process.

### SUMMARY

An objective of the present disclosure is to provide a culture capsule and a method for resuscitating bacteria in a VBNC state. In the present disclosure, a nutrient can be directly used or a water-soluble capsule storing the nutrient can be used to resuscitate bacteria in a VBNC state, such that the bacteria are restored to a culturable state, which is energy-saving, convenient, and fast, does not require secondary processing, and allows the goal of “awakening” simply and efficiently.

The present disclosure provides use of a nutrient in preparation of a resuscitating agent for resuscitating bacteria in a VBNC state, where the nutrient includes egg yolk lecithin and/or cholesterol.

The present disclosure provides a culture capsule for resuscitating bacteria in a VBNC state, including a nutrient and a water-soluble capsule shell encapsulating the nutrient, where the nutrient includes egg yolk lecithin and/or cholesterol.

In some embodiments, a material of the water-soluble capsule shell includes: a mixture of hydroxypropyl methylcellulose and gelatin, or a mixture of waxy rice starch and bone gelatin.

In some embodiments, the culture capsule includes 0.05 g to 0.06 g of the egg yolk lecithin and/or 0.10 g to 0.11 g of the cholesterol.

The present disclosure also provides use of a nutrient or the culture capsule described above in promotion of resuscitation of bacteria in a VBNC state at room temperature, where the nutrient includes egg yolk lecithin and/or cholesterol.

The present disclosure also provides a method for resuscitating bacteria in a VBNC state, including the following steps: mixing a nutrient or the culture capsule described above with a bacterial solution including the bacteria in the VBNC state, and statically cultivating a resulting mixture at room temperature,
where the nutrient includes egg yolk lecithin and/or cholesterol.

In some embodiments, when the nutrient is added alone, the nutrient is added per 20 mL of the bacterial solution as follows: more than 3 g of the egg yolk lecithin and/or more than 0.22 g of the cholesterol.

In some embodiments, when the culture capsule is added, one culture capsule is added per 20 mL of the bacterial solution.

In some embodiments, the bacteria in the bacterial solution have a concentration of no less than 3 log cell-forming unit (CFU)/mL.

In some embodiments, the static cultivation is conducted at 25±1° C. for 16 h to 24 h.

Beneficial effects: The present disclosure provides use of a nutrient in the preparation of a resuscitating agent for resuscitating bacteria in a VBNC state, where the nutrient includes egg yolk lecithin and/or cholesterol. In the examples, it has been confirmed through experiments that the nutrient, either added alone or encapsulated in a water-soluble capsule shell, can effectively, quickly, and simply promote the resuscitation of bacteria in a special dormant state.

In the present disclosure, the addition of the nutrient alone to resuscitate bacteria in a VBNC state has a higher cost than that of the addition of the nutrient encapsulated in the culture capsule. The encapsulation of the nutrient by a water-soluble capsule shell may significantly reduce the consumption of the nutrient, involves simple materials, convenient operations, and affordable prices, and perfectly avoids the shortcoming that the traditional medium needs to be sterilized. The culture capsule alone can be added to allow resuscitation. In addition, the operations of the present disclosure do not require additional professional devices such as a microbial incubator and a thermostatic shaker, and there is no need to consider the time of delivery of a sample to a testing laboratory, which is conducive to overcoming the shortcoming of lack of professional testing devices and personnel in remote areas and allowing the efficient “awakening” of dormant microorganisms in a sample.

### BRIEF DESCRIPTION OF THE DRAWINGS

FIGS. 1A-1C show pictures of the water-soluble culture capsules of the present disclosure, where a capsule with egg yolk lecithin encapsulated (FIG. 1A), a capsule with cholesterol encapsulated (FIG. 1i), and capsule shells (FIG. 1C) are shown; and the capsule shells all are cellulose capsule shells;

FIGS. 2A-2D show numbers of dead and viable bacteria in a bacterial solution including bacteria in a special dormant state under low-temperature and high-temperature treatments that are detected by flow cytometry, and a standard curve plotted accordingly according to a ratio of viable bacteria to dead bacteria (FIG. 2A), where a flow cytometry graph of total viable bacteria (FIG. 2B), a flow cytometry graph of total dead bacteria (FIG. 2C), and a flow cytometry graph of a treated bacterial solution (FIG. 2D) are shown; and in each of the flow cytometry graphs, P6 represents a zone of dead bacteria, 2,913 events are collected for P6 in FIG. 2D, with a mean fluoresce intensity of 1,828 in FL1 and 19,081 in FL3, P11 represents a zone of viable bacteria, 0 event is collected for P11 in FIG. 2D, and a zone between the two zones represents bacteria in a special dormant state;

FIGS. 3A-3I show colony pictures of Salmonella ATCC14028 in a special dormant state after being “awakened” through cultivation with a specified amount of a nutrient alone for 1 d to 7 d at room temperature (FIG. 3A), and flow cytometry graphs of a blank group every day, where flow cytometry graphs of the blank group on day 0, 1, 2, and up to day 7 (FIGS. 3B-3I) are shown sequentially, P6 represents a zone of dead bacteria, 2,182 (with a mean fluoresce intensity of 3,074 in FL1 and 37,423in FL3), 3,247 (with a mean fluoresce intensity of 1,695 in FL1 and 11,691 in FL3), 4,011 (with a mean fluoresce intensity of 1,777 in FL1 and 11,986 in FL3), 5,478 (with a mean fluoresce intensity of 2,003 in FL1 and 13,108 in FL3), 4,133 (with a mean fluoresce intensity of 1,951 in FL1 and 13,209 in FL3), 4,495 (with a mean fluoresce intensity of 1,423 in FL1 and 12,155 in FL3), 3,170 (with a mean fluoresce intensity of 937 in FL1 and 11,153 in FL3), 3,288 (with a mean fluoresce intensity of 2,536 in FL1 and 15,431 in FL3) events are collected for P6 in FIGS. 3B-3I, respectively; P11 represents a zone of viable bacteria, 0 events are collected for P11 in FIGS. 3B-3D and 3F-3G, 1 event is collected in FIG. 3E with a fluoresce intensity of 19,996 in FL1 and 5,340 in FL3; and a zone between the two zones represents bacteria in a special dormant state;

FIGS. 4A-4B show a total number of colonies of Salmonella ATCC14028 in a special dormant state after being “awakened” through cultivation with a waxy rice capsule at room temperature for 24 h in Example 2 (FIG. 4A) and a floating mass of a dissolved waxy rice capsule that hardly releases egg yolk lecithin (FIG. 4B);

FIG. 5 shows a total number of colonies of Salmonella ATCC14028 in a special dormant state after being “awakened” through cultivation for 24 h at room temperature with each of a fiber capsule including 0.11 g of cholesterol and a fiber capsule including 0.06 g of egg yolk lecithin and 0.11 g of cholesterol; and

FIG. 6 shows a total number of colonies of Salmonella ATCC14028 in a special dormant state after being “awakened” through cultivation for 16 h to 24 h at room temperature with each of a fiber capsule including 0.11 g of cholesterol and 0.06 g of egg yolk lecithin and a fiber capsule including 0.06 g of egg yolk lecithin.

### DETAILED DESCRIPTION OF THE EMBODIMENTS

The present disclosure provides use of a nutrient in preparation of a resuscitating agent for resuscitating bacteria in a VBNC state, where the nutrient includes egg yolk lecithin and/or cholesterol.

The egg yolk lecithin and the cholesterol of the present disclosure are common commercially-available products in the art, for example, the egg yolk lecithin can be egg yolk lecithin with a purity of 90% to 95% and the cholesterol can be cholesterol with a purity of 95% to 99%. The VBNC state of the present disclosure is also called a special dormant state. The nutrient can be directly added to a solution of bacteria in a VBNC state to promote resuscitation of the bacteria.

The nutrient of the present disclosure can be a single nutrient, a mixture of nutrients, or a water-soluble capsule encapsulating the nutrient. The Examples have confirmed that the water-soluble capsule has an excellent resuscitation effect, and a water-soluble capsule including egg yolk lecithin alone has a more significant resuscitation effect than both that including egg yolk lecithin and cholesterol and that including cholesterol alone.

The present disclosure provides a culture capsule for resuscitating bacteria in a VBNC state, including a nutrient and a water-soluble capsule shell encapsulating the nutrient, where the nutrient includes egg yolk lecithin and/or cholesterol.

The nutrient encapsulated in the culture capsule of the present disclosure includes preferably 0.05 g to 0.06 g of egg yolk lecithin and/or 0.10 g to 0.11 g of cholesterol, more preferably 0.06 g of egg yolk lecithin and/or 0.11 g of cholesterol, and most preferably 0.06 g of egg yolk lecithin.

In the present disclosure, the nutrient is encapsulated by a water-soluble capsule shell, and a material of the water-soluble capsule shell preferably includes: a mixture of hydroxypropyl methylcellulose and gelatin, or a mixture of waxy rice starch and bone gelatin. When the water-soluble capsule shell is prepared with the mixture of hydroxypropyl methylcellulose and gelatin, a preparation process is preferably as follows: first water is heated to 90° C., the gelatin is added, and after gelatin is dissolved, the hydroxypropyl methylcellulose is added to obtain a first mixture; the first mixture is continuously stirred and cooled to 60° C., then second water at 60° C. is added to obtain a second mixture, and the second mixture is continuously stirred thoroughly, then allowed to stand at 45° C. to remove bubbles, then placed in a mold, cooled, and demolded to obtain a fiber capsule shell. In the present disclosure, the water, the gelatin, and the hydroxypropyl methylcellulose are in a mass ratio of 100:0.4:12, and a mass ratio of the first water to the second water is 2:1. When the water-soluble capsule shell is prepared with the mixture of waxy rice starch and bone gelatin, a preparation process is preferably as follows: 95% (m/m) of the waxy rice starch and 5% (m/m) of the bone gelatin are mixed, and after the waxy rice starch is dissolved, a resulting mixture is placed in a mold, cooled, and demolded to obtain a waxy rice capsule shell. In the present disclosure, after the water-soluble capsule shell is prepared, a specified type of a nutrient can be added at a specified weight to the empty capsule shell to obtain the culture capsule of the present disclosure.

The present disclosure also provides use of a nutrient or the culture capsule described above in promotion of resuscitation of bacteria in a VBNC state at room temperature, where the nutrient includes egg yolk lecithin and/or cholesterol.

In the present disclosure, to promote the resuscitation of bacteria in a VBNC state, preferably, the nutrient or the culture capsule is adopted; more preferably, the culture capsule is adopted; further more preferably, a water-soluble capsule prepared with a fiber capsule shell and a nutrient is adopted; and most preferably, a water-soluble capsule prepared with a fiber capsule shell and egg yolk lecithin is adopted. The room temperature in the present disclosure preferably refers to 25±1° C.

The present disclosure also provides a method for resuscitating bacteria in a VBNC state, including the following steps: mixing a nutrient or the culture capsule described above with a bacterial solution including the bacteria in the VBNC state, and cultivating a resulting mixture statically at room temperature,
the nutrient includes egg yolk lecithin and/or cholesterol.

In an embodiment of the present disclosure, with Salmonella ATCC14028 as an example, it is confirmed that a minimum concentration of Salmonella ATCC14028 in a special dormant state to be resuscitated is about 3 log CFU/mL. In the present disclosure, to resuscitate Salmonella ATCC14028 in a special dormant state, when the nutrient is added alone, the nutrient added per 20 mL of the bacterial solution preferably includes: more than 3 g of the egg yolk lecithin and/or more than 0.22 g of the cholesterol; and when the culture capsule is added, one culture capsule is preferably added per 20 mL of the bacterial solution.

In the present disclosure, preferably, after the culture capsule is added to the bacterial solution, a resulting mixture is shaken until the culture capsule is dissolved and then subjected to static cultivation, where the static cultivation is conducted at preferably 25±1° C. for preferably 16 h to 24 h.

To further illustrate the present disclosure, the culture capsule and the method for resuscitating bacteria in a VBNC state according to the present disclosure are described in detail below with reference to examples, but these examples should not be construed as limiting the claimed scope of the present disclosure.

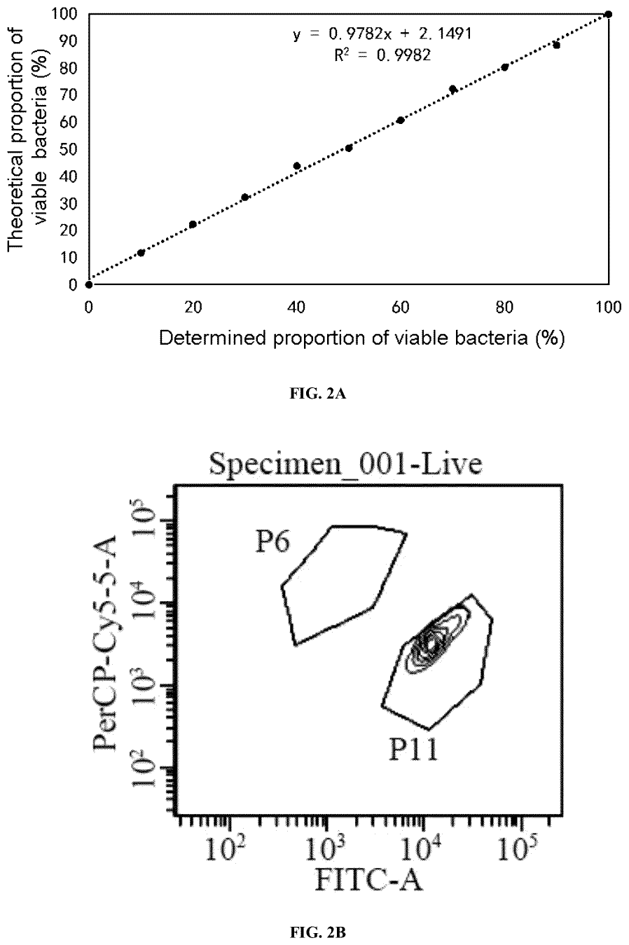

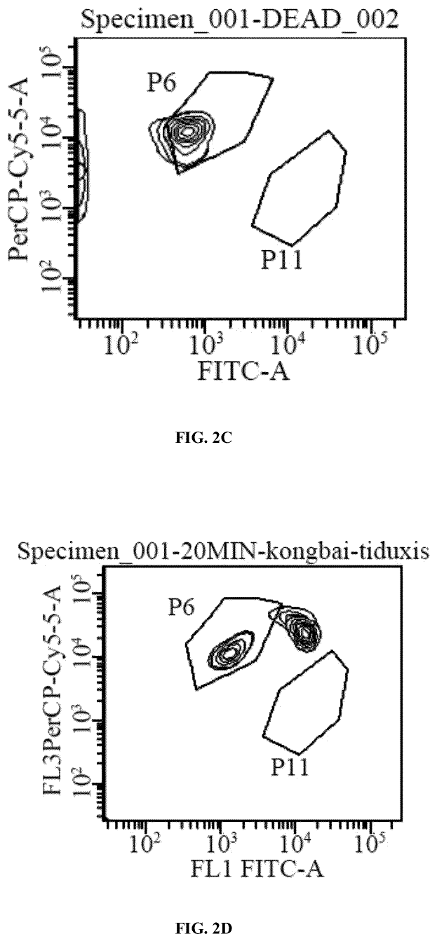

In the examples of the present disclosure, induced preparation of Salmonella ATCC14028 in a special dormant state: single colonies of Salmonella ATCC14028 are inoculated in a liquid medium and cultivated at 37° C. and 180 rpm to a late logarithmic stage; a resulting bacterial solution is centrifuged at 6,000 rpm for 10 min, and a resulting supernatant is discarded; a resulting precipitate is washed twice with phosphate buffered saline (PBS) and then re-dissolved with PBS for later use, and a resulting bacterial solution is serially diluted to a concentration of 1*10⁸ CFU/mL; 18 mL of PBS is added to a 50 mL centrifuge tube, the centrifuge tube is heated in a 60±2° C. water bath, and after the temperature in the centrifuge tube reaches 60±2° C., 2 mL of a 1*10⁸ CFU/mL bacterial solution is added to the centrifuge tube; a diluted bacterial solution is magnetically stirred and heated for 20 min, and then immediately taken out and placed in ice for later use; a cooled bacterial solution is centrifuged at 7,000 rpm for 10 min, a resulting supernatant is discarded, and PBS is added to 20 mL for re-dissolution; and the treated bacterial solution is subjected to staining with a Live/Dead BacLight kit, and then the number of viable bacteria in the treated bacterial solution is detected by flow cytometry. When the number of viable bacteria in the treated bacterial solution is 0, it indicates that the Salmonella ATCC14028 has entered a special dormant state, as shown in FIG. 2A. 100 μL of the bacterial solution is evenly coated on a plate count agar (PCA) counting plate by a plate counting method, and the PCA counting plate is invertedly placed in an incubator and incubated anaerobically or aerobically at 37° C. for 18 h to 24 h. After the cultivation is completed, no bacterial colonies grow.

Flow cytometry graphs of total dead/viable bacteria: 1 mL of a bacterial solution heat-treated at 95° C. for 10 min is taken and subjected to staining with a component A (1.5 μL) alone in a kit, incubated in the dark for 15 min, and then detected by flow cytometry, and a zone of total dead bacteria is marked, as shown in FIGS. 2B-2D. 1 mL of an untreated bacterial solution is subjected to staining with a component B (1.5 μL) alone, incubated in the dark for 15 min, and then detected by flow cytometry, and a zone of total viable bacteria is marked, as shown in FIG. 2. Theoretical and actual percentages of viable bacteria in the treated bacterial solution are determined by flow cytometry in ratios of 0:100, 10:90, 20:80, 30:70, 40:60, 50:50, 60:40, 70:30, 80:20, 90:10, and 100:0, and a standard curve is plotted, as shown in FIG. 2A.

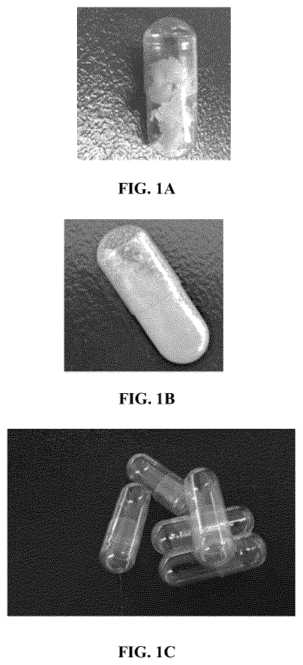

Preparation of culture capsules: ⅔ of 100 mL of water is heated to 90° C., 0.4 g of gelatin is added, and after the gelatin is dissolved, 12 g of hydroxypropyl methylcellulose is added to obtain a first mixture; the first mixture is continuously stirred and cooled to 60° C., and then ⅓ of 100 mL of water at 60° C. is added to obtain a second mixture; and the second mixture is thoroughly stirred, then allowed to stand at 45° C. to remove bubbles, placed in a mold, cooled, and demolded to obtain a fiber capsule shell. 95% of waxy rice starch and 5% of bone gelatin are mixed, and after the waxy rice starch is dissolved, a resulting mixture is placed in a mold, cooled, and demolded to obtain a waxy rice capsule shell. 0.06 g of egg yolk lecithin or 0.11 g of cholesterol is added to an empty capsule shell to obtain a culture capsule, as shown in FIGS. 1A-1C.

Effect of “awakening” bacteria in a special dormant state is determined by a plate counting method, and a specific process is as follows: one sterile water-soluble culture capsule with egg yolk lecithin encapsulated and one sterile water-soluble culture capsule with cholesterol encapsulated each are placed in 20 mL of a bacterial solution including Salmonella ATCC14028 in a special dormant state, and the bacterial solution is shaken until a capsule shell is dissolved and then allowed to stand at room temperature for 16 h to 24 h; 100 μL of the bacterial solution in which bacteria are “awakened” for 16 h to 24 h is evenly coated on a PCA counting plate, then the PCA counting plate is invertedly placed in an incubator and incubated anaerobically or aerobically at 37° C. for 18 h to 24 h, and then the growth of colonies is observed. Compared with a blank bacterial sample in which no water-soluble capsule is added, colonies grow in a bacterial sample in which a capsule is added, indicating that “awakened” colonies grow from bacteria in a special dormant state.

### Example 1

A specified amount (more than 3 g) of egg yolk lecithin and a specified amount (more than 0.22 g) of cholesterol (which were not encapsulated by a capsule) were added to “awaken” Salmonella in a special dormant state.

A bacterial solution in which bacteria were induced to enter a special dormant state was taken and subjected to staining with a Live/Dead BacLight kit, and the number of viable bacteria in the bacterial solution was detected by flow cytometry. When the number of viable bacteria was 0, as shown in FIG. 2A, it indicated that the Salmonella ATCC14028 had entered a special dormant state.

100 μL of the bacterial solution was evenly coated on a PCA counting plate by a plate counting method, and the PCA counting plate was invertedly placed in an incubator and incubated anaerobically or aerobically at 37° C. for 18 h to 24 h. After the cultivation was completed, no bacterial colonies grew. Egg yolk lecithin and cholesterol each were added to 20 mL of a bacterial solution of Salmonella ATCC14028 in a special dormant state, and then the bacterial solution was statically incubated in an incubator at 37° C. for 1 d to 7 d; and 1 mL of an “awakened” bacterial solution was taken and inoculated on a plate by a pour-plate method, the plate was invertedly placed in an incubator and incubated anaerobically or aerobically at 37° C. for 18 h to 24 h, and the growth of colonies was observed.

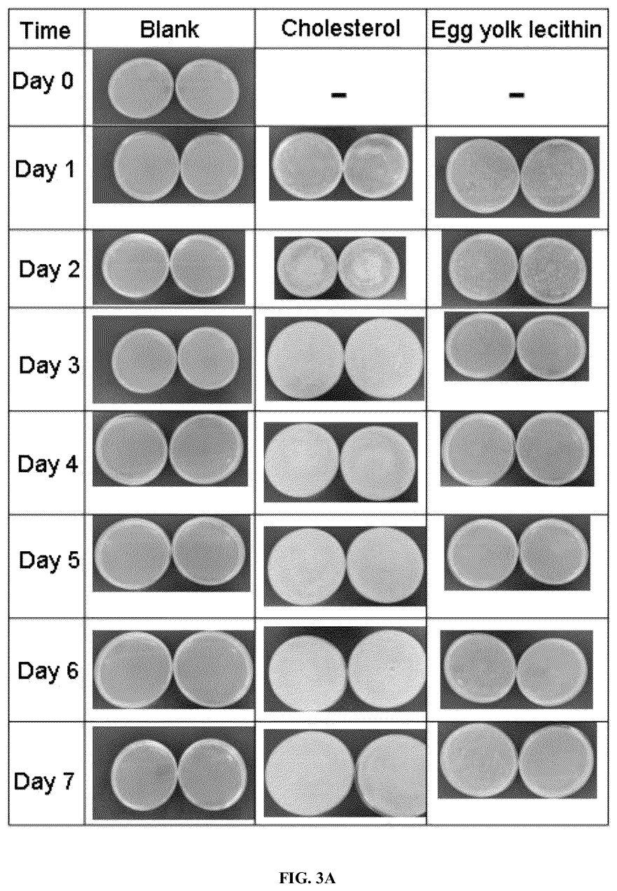

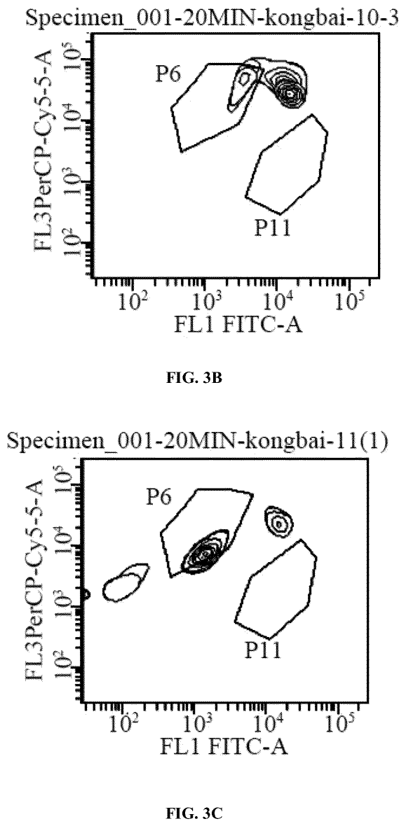

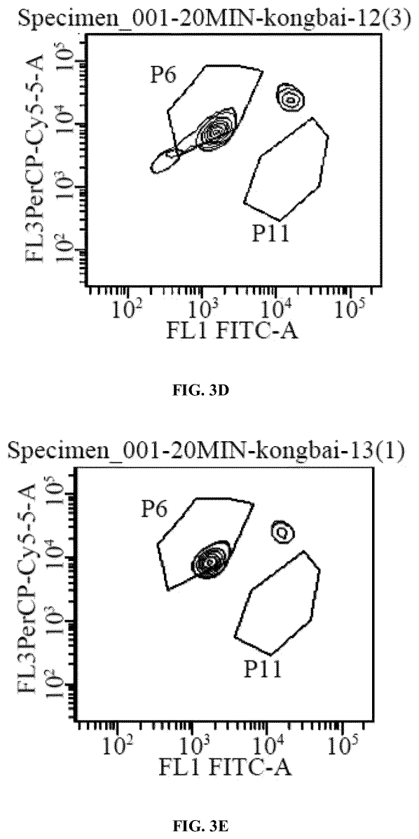

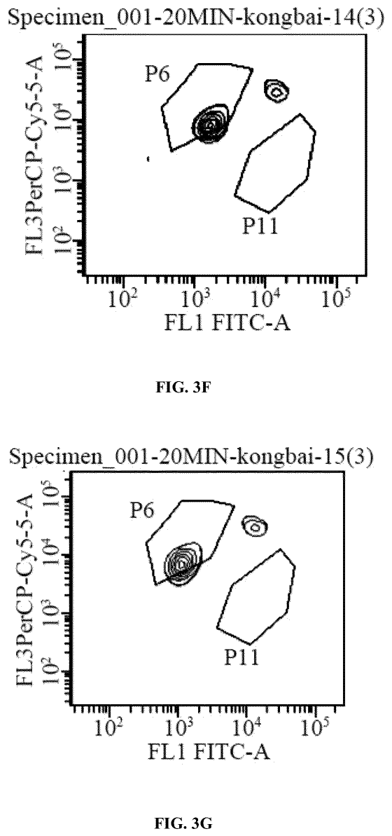

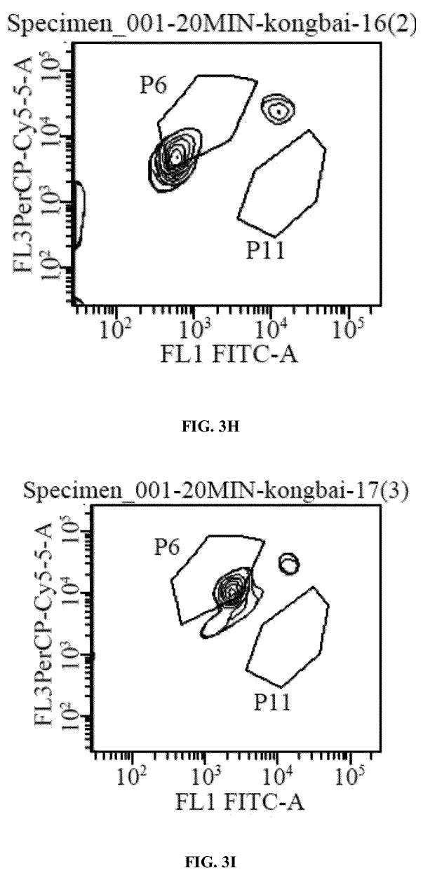

Experimental results are shown in FIGS. 3A-3I, and it can be seen that both cholesterol and egg yolk lecithin can “awaken” bacteria in a special dormant state. Given the cost and improvement of utilization rate of the nutrient, self-made water-soluble capsules in which the nutrient content decreased proportionally in the present disclosure were used in the subsequent experiments.

### Example 2

1. A waxy rice capsule including 0.06 g of egg yolk lecithin was added to “awaken” Salmonella in a special dormant state.

Preparation of the culture capsule: 0.06 g of egg yolk lecithin was added to an empty waxy rice capsule.

A bacterial solution in which bacteria were induced to enter a special dormant state was taken and subjected to staining with a Live/Dead BacLight kit, and the number of viable bacteria in the bacterial solution was detected by flow cytometry. When the number of viable bacteria in the bacterial solution was 0, it indicated that the Salmonella ATCC14028 had entered a special dormant state. 100 μL of the bacterial solution was evenly coated on a PCA counting plate by a plate counting method, and the PCA counting plate was invertedly placed in an incubator and incubated anaerobically or aerobically at 37° C. for 18 h to 24 h. After the cultivation was completed, no bacterial colonies grew.

One sterile water-soluble culture waxy rice capsule with egg yolk lecithin encapsulated was placed in 20 mL of a bacterial solution including Salmonella ATCC14028 in a special dormant state, and the bacterial solution was shaken until the capsule shell was dissolved and then allowed to stand at room temperature for 24 h; and 100 μL of the bacterial solution in which bacteria were “awakened” for 16 h to 24 h was evenly coated on a PCA counting plate, then the PCA counting plate was invertedly placed in an incubator and incubated anaerobically or aerobically at 37° C. for 18 h to 24 h, and then the growth of colonies was observed.

2. A waxy rice capsule including 0.11 g of cholesterol was added to “awaken” Salmonella in a special dormant state.

Preparation of the culture capsule: 0.11 g of cholesterol was added to an empty waxy rice capsule.

A bacterial solution in which bacteria were induced to enter a special dormant state was taken and subjected to staining with a Live/Dead BacLight kit, and the number of viable bacteria in the bacterial solution was detected by flow cytometry. If the number of viable bacteria in the bacterial solution was 0, it indicated that the Salmonella ATCC14028 had entered a special dormant state. 100 μL of the bacterial solution was evenly coated on a PCA counting plate by the plate counting method, and the PCA counting plate was invertedly placed in an incubator and incubated anaerobically or aerobically at 37° C. for 18 h to 24 h. After the cultivation was completed, no bacterial colonies grew. One sterile water-soluble culture waxy rice capsule with cholesterol encapsulated was placed in 20 mL of a bacterial solution including Salmonella ATCC14028 in the special dormant state, and the bacterial solution was shaken until the capsule shell was dissolved and then allowed to stand at room temperature for 24 h; and 100 μL of the bacterial solution in which bacteria were “awakened” for 16 h to 24 h was evenly coated on a PCA counting plate, then the PCA counting plate was invertedly placed in an incubator and incubated anaerobically or aerobically at 37° C. for 18 h to 24 h, and then the growth of colonies was observed.

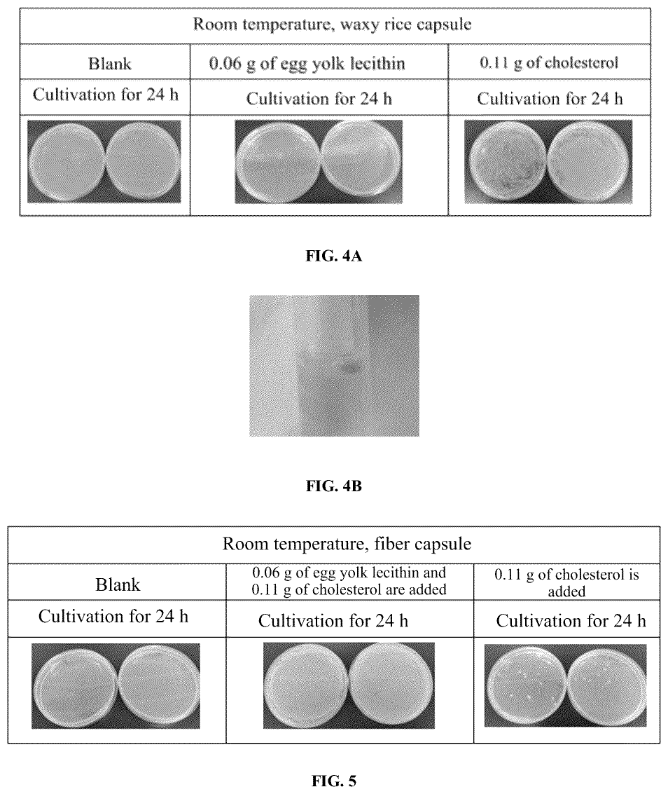

Results are shown in FIGS. 4A-4B. The egg yolk lecithin-containing culture waxy rice capsule failed to “awaken” bacteria in the bacterial solution, while the cholesterol-containing culture waxy rice capsule successfully “awakened” bacteria in the bacterial solution. The above situation occurred because the egg yolk lecithin could not be released due to characteristics of the waxy rice capsule itself. Therefore, the waxy rice capsule was abandoned in the subsequent experiments. The blank group was sterile, that is, bacteria in a bacterial solution in the blank group were in a special dormant state before the nutrient was added.

### Example 3

A fiber capsule including 0.11 g of cholesterol, a fiber capsule including 0.06 g of egg yolk lecithin, and a fiber capsule including 0.11 g of cholesterol and 0.06 g of egg yolk lecithin each were added to “awaken” Salmonella in a special dormant state.

Preparation of culture capsules: 0.11 g of cholesterol was added to an empty fiber capsule shell to prepare the cholesterol-containing fiber capsule; 0.06 g of egg yolk lecithin was added to an empty fiber capsule shell to prepare the egg yolk lecithin-containing capsule; and 0.06 g of egg yolk lecithin and 0.11 g of cholesterol were added to an empty fiber capsule shell to prepare the egg yolk lecithin and cholesterol-containing capsule.

A bacterial solution in which bacteria were induced to enter a special dormant state was taken and subjected to staining with a Live/Dead BacLight kit, and the number of viable bacteria in the bacterial solution was detected by flow cytometry. When the number of viable bacteria in the bacterial solution was 0, it indicated that the Salmonella ATCC14028 had entered a special dormant state. 100 μL of the bacterial solution was evenly coated on a PCA counting plate by the plate counting method, and the PCA counting plate was invertedly placed in an incubator and incubated anaerobically or aerobically at 37° C. for 18 h to 24 h. After the cultivation was completed, no bacterial colonies grew. One sterile water-soluble culture fiber capsule with cholesterol encapsulated, one sterile water-soluble culture fiber capsule with egg yolk lecithin encapsulated, and one sterile water-soluble culture fiber capsule with both egg yolk lecithin and cholesterol encapsulated each were placed in 20 mL of a bacterial solution including Salmonella ATCC14028 in a special dormant state, and the bacterial solution was shaken until a capsule shell was dissolved and then allowed to stand at room temperature for 16 h to 24 h; and 100 μL of the bacterial solution in which bacteria were “awakened” for 16 h to 24 h was evenly coated on a PCA counting plate, then the PCA counting plate was invertedly placed in an incubator and incubated anaerobically or aerobically at 37° C. for 18 h to 24 h, and then the growth of colonies was observed.

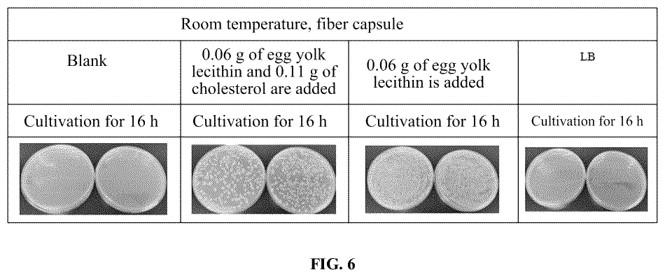

Experimental results are shown in FIG. 5 and FIG. 6. The blank group was sterile, that is, bacteria in the bacterial solution of the blank group were in a special dormant state before the nutrient is added. The addition of both 0.06 g of egg yolk lecithin and 0.11 g of cholesterol had a better effect of “awakening” bacteria in the special dormant state than the addition of 0.11 g of cholesterol alone, which may be due to an inhibitory effect of a fiber capsule on cholesterol. After the culture capsule was added to the treated bacterial solution and the bacterial solution was statically cultivated at room temperature for 16 h, bacteria in the bacterial solution can be “awakened”, and the effect was more significant when 0.06 g of egg yolk lecithin was added. Under the same conditions, bacteria in the treated bacterial solution in which Luria-Bertani (LB) broth was added were not “awakened”. In summary, most preferably, a water-soluble fiber capsule including 0.06 g of egg yolk lecithin was selected.

### Example 5

The minimum “awakenable” concentration was determined

Single colonies of Salmonella ATCC14028 were inoculated in a liquid medium and cultivated at 37° C. and 180 rpm to a late logarithmic stage; a resulting bacterial solution was centrifuged at 6,000 rpm for 10 min, and a resulting supernatant was discarded; a resulting precipitate was washed twice with PBS and then re-dissolved with PBS for later use, and a resulting bacterial solution was serially diluted to a concentration of 1*10⁸ CFU/mL; 18 mL of PBS was added to a 50 mL centrifuge tube, the centrifuge tube was heated in a 60±2° C. water bath, and after the temperature in the centrifuge tube reached 60±2° C., 2 mL of a 1*10⁸ CFU/mL bacterial solution was added to the centrifuge tube; the diluted bacterial solution was magnetically stirred and heated for 20 min, and then immediately taken out and placed in ice for later use; the cooled bacterial solution was centrifuged at 7,000 rpm for 10 min, a resulting supernatant was discarded, and PBS was added to 20 mL for re-dissolution; and the treated bacterial solution was subjected to staining with a Live/Dead BacLight kit, and then the number of viable bacteria in the treated bacterial solution was detected by flow cytometry, where it was determined that bacteria in the treated bacterial solution entered a special dormant state.

The bacterial solution in which bacteria were induced to enter a special dormant state was serially diluted to concentrations of 10⁶ CFU/mL, 10⁵ CFU/mL, 10⁴ CFU/mL, and 10³ CFU/mL; and 100 μL of each of diluted bacterial solutions (concentrations: 105 CFU/mL, 10⁴ CFU/mL, 10³ CFU/mL, and 10² CFU/mL) was taken and evenly coated on a PCA counting plate, then cultivated in an incubator for 18 h to 24 h, and readings were recorded. Results showed that Salmonella grew well at concentrations of 10⁵ CFU/mL, 10⁴ CFU/mL, and 10³ CFU/mL, and bacteria did not grow on a plate with a concentration of 10² CFU/mL. Taking the test results of flow cytometry into consideration, it is determined that the minimum concentration is about 3 log CFU/mL for resuscitation of Salmonella ATCC14028 in a special dormant state.

Although the present disclosure has been described in detail through the above examples, the examples are merely some rather than all of the embodiments of the present disclosure. All other embodiments obtained by a person based on these examples without creative efforts shall fall within the protection scope of the present disclosure.

What is claimed is:

1. A method for resuscitating bacteria in a viable but non-culturable (VBNC) state, comprising administering a nutrient to the bacteria in the VBNC state, wherein the nutrient comprises egg yolk lecithin and/or cholesterol.

2. A culture capsule for resuscitating bacteria in a VBNC state, comprising a nutrient and a water-soluble capsule shell encapsulating the nutrient, wherein the nutrient comprises egg yolk lecithin and/or cholesterol.

3. The culture capsule according to claim 2, wherein a material of the water-soluble capsule shell comprises: a mixture of hydroxypropyl methylcellulose and gelatin, or a mixture of waxy rice starch and bone gelatin.

4. The culture capsule according to claim 2, wherein the culture capsule comprises 0.05 g to 0.06 g of the egg yolk lecithin and/or 0.10 g to 0.11 g of the cholesterol.

5. A method for resuscitating bacteria in a VBNC state, comprising the following steps: mixing a nutrient or the culture capsule according to claim 2 with a bacterial solution comprising the bacteria in the VBNC state, and statically cultivating a resulting mixture at room temperature, wherein the nutrient comprises egg yolk lecithin and/or cholesterol.

6. The method according to claim 5, wherein when the nutrient is added alone, the nutrient added per 20 mL of the bacterial solution includes: more than 3 g of the egg yolk lecithin and/or more than 0.22 g of the cholesterol.

7. The method according to claim 5, wherein when the culture capsule is added, one culture capsule is added per 20 mL of the bacterial solution.

8. The method according to claim 6, wherein a concentration of the bacteria in the bacterial solution is no less than 3 log cell-forming unit (CFU)/mL.

9. The method according to claim 5, wherein the static cultivation is conducted at 25±1° C. for 16 h to 24 h.

10. The method according to claim 5, wherein a material of the water-soluble capsule shell comprises: a mixture of hydroxypropyl methylcellulose and gelatin, or a mixture of waxy rice starch and bone gelatin.

11. The method according to claim 5, wherein the culture capsule comprises 0.05 g to 0.06 g of the egg yolk lecithin and/or 0.10 g to 0.11 g of the cholesterol.

12. The method according to claim 8, wherein when the culture capsule is added, one culture capsule is added per 20 mL of the bacterial solution.
